(19) United States
(12) Patent Application Publication
(10) Pub. No.: US 2025/0288801 A1
(43) Pub. Date: Sep. 18, 2025

(54) MASSAGE DEVICE USING MICROCURRENT

(71) Applicant: GSSEM GLOBAL CO.,LTD., Seoul (KR)

(72) Inventor: Dae Hyun KIM, Seoul (KR)

(21) Appl. No.: 19/121,258

(22) PCT Filed: Aug. 11, 2023
(86) PCT No.: PCT/KR2023/011988

§ 371 Date: Apr. 15, 2025

(30) Foreign Application Priority Data
Aug. 12, 2022 (KR) 10-2022-0101338
May 16, 2023 (KR) 10-2023-0062763

(51) Int. Cl. A61N 1/32; A61H 23/02; A61N 5/06

(52) U.S. Cl. CPC A61N 1/327; A61H 23/0254; A61H 2201/0207; A61H 2201/10; A61H 2201/105; A61H 2201/5058; A61N 5/062; A61N 2005/0651

(57) ABSTRACT

The present invention relates to a massage device using microcurrent. The massage device comprises: a container accommodating a liquid material; and a massage unit which applies the liquid material and applies physical and electrical stimuli onto a contact site to massage skin or muscles. According to the present invention, first protrusions and second protrusions contact the body to apply microcurrent to a contact site, thereby allowing the active ingredient included in the liquid material to penetrate and be absorbed deep into the skin and thus maximizing cosmetic and treatment effects. In addition, by removal of the remaining liquid material present on the upper surface or the inside of the massage unit through vibration or heat, it is possible to prevent hygiene problems from occurring.

### TECHNICAL FIELD

The present invention relates to a massage device. More particularly, the present invention relates to a massage device using a microcurrent that may improve absorption and massage effects of an active ingredient contained in an applied liquid material by inducing a microcurrent to flow through a contact area.

### BACKGROUND ART

In modern times, as interest in beauty and physical health increases, liquid materials such as gels or creams containing ingredients that maintain and improve health of skin or muscles are being developed, and these liquid materials are being used in massage.

Meanwhile, the liquid materials are generally absorbed by tapping or rubbing with the hands after coating, but this method has a limitation in deeply absorbing active ingredients contained in the liquid materials into the skin, and in an applying and absorbing process, the liquid materials may be excessively absorbed, resulting in poor effectiveness.

Further, when a user uses a massage device alone, there is a limited range in which the liquid materials can be applied or massage can be performed, there is a limitation in stimulating the skin while maintaining a constant pressure for a long time, and accordingly, sufficient stimulation cannot be delivered to the skin or muscles, which may reduce effectiveness thereof.

To solve these problems, related document 1 and related document 2 propose massagers that help blood circulation, reduce fatigue, and at the same time, absorb applied liquid materials by applying various types of physical stimuli, e.g., pressing or tapping a portion where the liquid materials are applied.

However, the massage devices used in related document 1 and related document 2 simply press a massage area using an equipped roller or equipped massage ball, and thus the user should continuously apply a force.

Further, when simply spreading the applied liquid materials on the skin using the roller or the massage ball, a long time is required for deeply absorbing the active ingredients contained in the liquid materials into the skin and exhibiting an effect, and thus the massage device is inefficient. Further, hygiene problems in which bacteria grow or contaminants are stuck to the liquid materials that are not absorbed to the skin and hardened on a surface of the roller or the massage ball may occur.

### DETAILED DESCRIPTION OF INVENTION

### Technical Problem

A technical problem of the present invention is to prevent an effect from decreasing due to an active ingredient included in a liquid material not being deeply absorbed into the skin.

Another technical problem of the present invention is to prevent loss occurring because a liquid material is excessively absorbed in a hand in a process of applying and absorbing the liquid material to a skin.

Still another technical problem of the present invention is to prevent an effect from being degraded due to sufficient stimulation not being transmitted to the skin or muscles during a process of performing a massage.

### Technical Solution

One aspect of the present invention to solve the above problems provides a massage device including a protrusion part including a plurality of first protrusions each having an upper end that is partially exposed through an upper surface of a massager and generates a microcurrent and a lower end that is provided inside the massager to have a first height from the upper surface and a plurality of second protrusions each having an upper end that is partially exposed through the upper surface of the massager and generates a microcurrent and a lower end that is provided inside the massager to have a second height from the upper surface, a first conductor having a plate shape, which is provided in contact with the lower ends of the plurality of first protrusions and transmits a current supplied from a controller to the plurality of first protrusions, and a second conductor having a plate shape, which is provided in contact with the lower ends of the plurality of second protrusions and transmits a current supplied from the controller to the plurality of second protrusions, wherein the first height and the second height are different heights.

Further, the massage device may further include a separation layer that is provided between the first conductor and the second conductor and insulates the first conductor from the second conductor while maintaining a constant gap between the first conductor and the second conductor.

Further, the controller may include a sensor that measures an intensity of a current applied to the protrusion part, compare a current value measured by the sensor based on a pre-stored reference current value to calculate an area of a contact area in contact with the protrusion part including the plurality of protrusions, and control an intensity of a voltage according to the area of the contact area.

Further, the massage device may include a vibration motor that generates vibration to stimulate a contact area with a constant pressure and removes a residual liquid material with which an upper surface of the massager is coated after use of the massager.

Further, the massage device may include a heating part that generates heat to promote absorption of the liquid material and prevents the liquid material remaining on an upper surface or an inside of the massager from solidifying after use of the massager.

Further, the first height may be greater than the second height.

Further, a diameter of the first protrusion may be greater than a diameter of the second protrusion.

Further, an outlet through which the liquid material is discharged may be formed on an upper surface of the massager, and the massager may include a guide part having a tubular shape, which passes through an inside of the massager, is connected to the container, and delivers the liquid material from the container to the outlet.

### Advantageous Effects

According to the present invention, an active ingredient contained in a liquid material is absorbed deep into the skin through electrical stimulation using a microcurrent, and thus an effect thereof can be maximized.

According to the present invention, a contact area is stimulated with a constant pressure through vibration during a coating and absorbing process of a liquid material, and thus a massage effect can be increased.

According to the present invention, a residual liquid material present on an upper surface or the inside of a massager through vibration or heating is removed, and thus hygiene problems can be prevented.

### DESCRIPTION OF DRAWINGS

FIG. 1 is a perspective view of a massage device according to an embodiment of the present invention.

FIG. 2 is an exploded perspective view illustrating a massager according to the embodiment of the present invention.

FIG. 3 is an exploded perspective view illustrating a head part according to the embodiment of the present invention.

FIG. 4 is a cross-sectional view of the head part according to the embodiment of the present invention.

### BEST MODE

A massage device including a massager that discharges a liquid material and a container in which the liquid material is accommodated includes:
a protrusion part including a plurality of first protrusions each having an upper end that is partially exposed through an upper surface of the massager and generates a microcurrent and a lower end that is provided inside the massager to have a first height from the upper surface and a plurality of second protrusions each having an upper end that is partially exposed through the upper surface of the massager and generates a microcurrent and a lower end that is provided inside the massager to have a second height from the upper surface;
a first conductor having a plate shape, which is provided in contact with the lower ends of the plurality of first protrusions and transmits a current supplied from a controller to the plurality of first protrusions; and
a second conductor having a plate shape, which is provided in contact with the lower ends of the plurality of second protrusions and transmits a current supplied from the controller to the plurality of second protrusions,
wherein the first height and the second height are different heights.

### Modes of the Invention

Hereinafter, embodiments of the present invention will be described in detail with reference to the accompanying drawings so that those skilled in the art to which the present invention pertains may easily implement the present invention. However, the present invention may be implemented in various different forms and is not limited to the embodiments described herein. Further, in order to clearly disclose the present invention in the drawings, parts irrelevant to the present invention will be omitted, and in the drawings, the same or similar reference numerals indicate the same or similar components.

The purpose and effect of the present invention may be naturally understood from or made clearer by the following description, and the purpose and effect of the present invention are not limited only to the following description.

The purpose, features, and advantages of the present invention will become more apparent from the following detailed description. Further, in the description of the present invention, when it is determined that the detailed description of widely known related technologies may make the subject matter of the present invention unclear, the detailed description will be omitted.

Hereinafter, embodiments of the present invention will be described in detail with reference to the accompanying drawings.

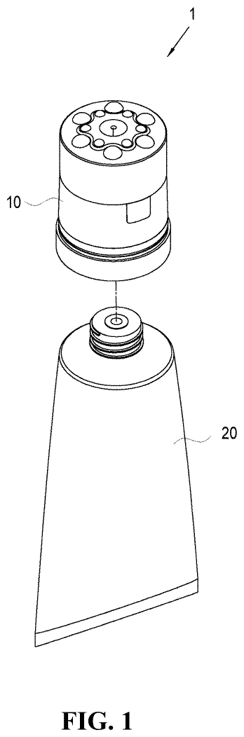

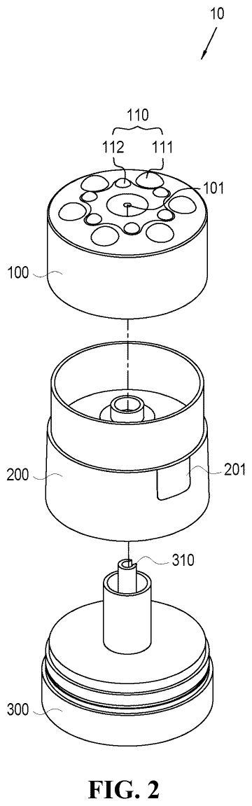

FIG. 1 is a perspective view of a massage device 1 according to an embodiment of the present invention, and FIG. 2 is an exploded perspective view illustrating a massager 10 according to the embodiment of the present invention.

Referring to FIGS. 1 and 2, the present invention relates to a massage device 1 including a massager 10 using which a body is coated with a liquid material while in contact with the body and which massages a contact area through physical and electrical stimulation and a container 20 that accommodates the liquid material, is coupled to a lower portion of the massager 10, and supplies the liquid material.

The massager 10 includes a tubular guide part 310 that passes through an inside thereof, is connected to the container 20, and delivers the liquid material from the container 20, and the liquid material delivered through the guide part 310 is discharged through an outlet 101 formed on an upper surface of the massager 10.

In this case, the massager 10 and the container 20 are provided in a form that may be coupled or separated, the effect is increased by performing the massage while applying the liquid material, and at the same time, when all the liquid material is exhausted, the massage device may be used after replacing only the container 20, and thus cost loss can be prevented.

In the present invention, the massager 10 is provided in a cylindrical shape and divided into a head part 100, a body part 200, and a coupling part 300, and the head part 100, the body part 200, and the coupling part 300 are described based on a form that may be coupled or separated. However, the present invention is not limited thereto and may be provided in various shapes and forms that may be easily gripped by a user, may be coupled to the container 20 to discharge and apply the liquid material onto the contact area, and at the same time, may perform the massage.

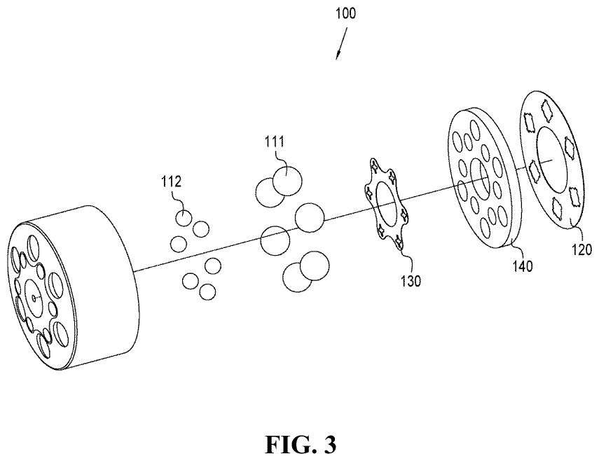

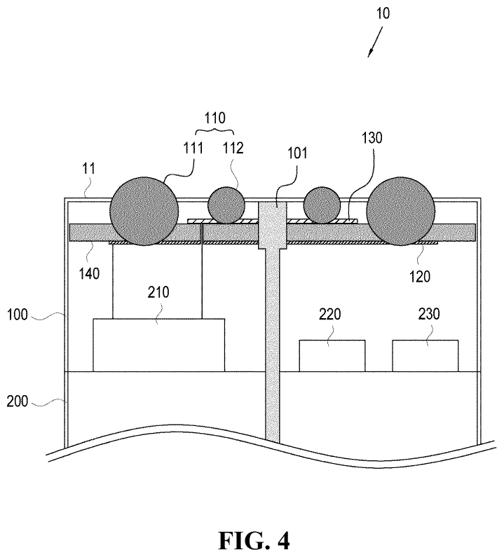

Referring to FIGS. 3 and 4, the head part 100 is a component for discharging the liquid material through the outlet 101 formed on an upper surface thereof, applying the liquid material onto the contact area, and at the same time, applying the physical and electrical stimulation, and may include a protrusion part 110, a first conductor 120, a second conductor 130, and a separation layer 140.

The protrusion part 110 includes a plurality of first protrusions 111 and a plurality of second protrusions 112 and is a component for massaging the contact area at a predetermined pressure while in contact with the body, at the same time, applying electrical stimulation, and thus promoting absorption of the liquid material and increasing a massage effect.

In detail, the first protrusions 111 and the second protrusions 112 are made of a conductive material, receive a current from a controller 210, which will be described below, apply a microcurrent to the contact area while in contact with the body, cause an active ingredient contained in the liquid material to penetrate deeply into the skin to promote absorption, and at the same time, apply electrical and physical stimulation to serve to massage the skin and muscles.

In detail, the head part 100 includes the plurality of first protrusions 111 and the plurality of second protrusions 112, and the first protrusions 111 and the second protrusions 112 receive a current from the controller 210 and apply the microcurrent to the contact area. In this case, the first protrusion 111 and the second protrusion 112 may be formed to have either a +pole or a −pole, and the microcurrent applied to the contact area by the first protrusion 111 and the second protrusion 112 stimulates and massages the skin or muscles, at the same time, causes the active ingredient contained in the liquid material to be deeply absorbed into the skin, and thus promotes muscle pain relief or skin regeneration.

In more detail, lower ends of the first protrusion 111 and the second protrusion 112 are exposed inside the massager 10 to receive a current from the controller 210 provided inside the massager 10. In detail, the lower ends of the plurality of first protrusions 111 and the plurality of second protrusions 112 are partially exposed inside the massager 10 and electrically connected to the controller 210 to receive a current from the controller 210, and upper ends thereof are partially exposed through an upper surface 11 of the massager 10 to apply the microcurrent to the contact area.

Preferably, the lower end of the first protrusion 111 and the lower end of the second protrusion 112 are formed to have different heights inside the massager 10 and are physically separated therefrom, and thus a current may flow normally, and at the same time, incidents such as fires due to mutual contact can be prevented. In detail, the lower end of the first protrusion 111 may have a first height h1 from the upper surface 11 inside the massager 10, the lower end of the second protrusion 112 may have a second height h2 from the upper surface 11 inside the massager 10, and the first height h1 may be formed to be greater than the second height h2.

In more detail, the first height h1 may be a height from the upper surface 11 of the massager 10 to a highest point at the lower end of the first protrusion 111, the second height h2 may be a height from the upper surface 11 of the massager 10 to a highest point at the lower end of the second protrusion 112, the first height h1 may be provided to be greater than the second height h2, and thus the first protrusion 111 and the second protrusion 112 may be spaced a predetermined height from each other inside the massager 10 to receive a current from the controller 210.

Further, a diameter of the first protrusion 111 may be formed to be greater than a diameter of the second protrusion 112, the plurality of first protrusions 111 and the plurality of second protrusions 112 may be provided to cross each other, the protrusion part 110 may include more protrusions in a small area, and thus the microcurrent may be applied to the contact area more efficiently.

FIGS. 3 and 4 illustrate that the first protrusion 111 and the second protrusion 112 have a spherical shape, but the present invention is not limited thereto, and the first protrusion 111 and the second protrusion 112 may be implemented in various shapes that apply the microcurrent to the contact area and apply physical stimulation.

The first conductor 120 and the second conductor 130 are components that are in contact with the first protrusion 111 and the second protrusion 112, respectively, and transmit a current supplied from the controller 210.

In detail, the first conductor 120 is provided in a plate shape, is formed in contact with surfaces of the lower ends of the plurality of first protrusions 111, and thus serves to simultaneously transmit the current supplied from the controller 210 to the plurality of first protrusions 111. The second conductor 130 is provided in a plate shape, is formed in contact with surfaces of the lower ends of the plurality of second protrusions 112, and thus serves to simultaneously transmit the current supplied from the controller 210 to the plurality of second protrusions 112.

It is preferable that the first conductor 120 and the second conductor 130 be spaced a predetermined height from each other such that the first conductor 120 and the second conductor 130 are not in contact with each other. In detail, it is more preferable that the second conductor 130 be formed in a disk shape, further protrude in a predetermined shape in a direction in which the second protrusion 112 is located, and not be in contact with the first protrusion 111. Further, the first conductor 120 and the second conductor 130 may further include contact protrusions protruding a predetermined height in a direction in which a power supply unit is positioned to receive current from the controller 210. Further, it is preferable that the first conductor 120 and the second conductor 130 be provided in an annular shape having a hollow center with the guide part 310 formed therebetween to deliver the liquid material.

The separation layer 140 is a component that prevents the first conductor 120 and the second conductor 130 from coming into contact with each other. In detail, the separation layer 140 is provided between the first conductor 120 and the second conductor 130, insulates the first conductor 120 from the second conductor 130 while maintaining a constant gap between the first conductor 120 and the second conductor 130, and at the same time, supports the first conductor 120 and the second conductor 130 such that the first conductor 120 and the second conductor 130 do not deviate from original positions thereof, and thus serves to maintain a state in which the first conductor 120 and the second conductor 130 are in contact with the first protrusion 111 and the second protrusion 112. It is preferable that the separation layer 140 be formed of a non-conductive material and prevent the first conductor 120 and the second conductor 130 from being electrically connected to each other. Further, it is preferable that the separation layer 140 be provided in an annular shape having a hollow center with the guide part 310 formed therebetween to deliver the liquid material.

The body part 200 may be a component for controlling an operation of the massager 10, a charging terminal 201 and a power switch (not illustrated) may be formed on one side of an outer circumferential surface of the body part 200, a space having a predetermined size may be formed inside the body part 200, and the body part 200 may include the controller 210, a vibration motor 220, and a heating part 230.

The charging terminal 201 may be a component for receiving power while in contact with an external power terminal, the power transmitted from the outside is transmitted to the controller 210, which will be described below, and the controller 210 may control vibration and heat by applying a current to the vibration motor 220 and the heating part 230, which will be described below.

Meanwhile, an internal space of the body part 200 may be further provided with a rechargeable battery, and the rechargeable battery may be electrically connected to the controller 210, store a current applied from the controller 210, and thus supply a current to the controller 210, while not connected to an external power terminal, and thus the controller 210 may control the vibration motor 220 and the heating part 230.

The power switch may be a component for turning the massager 10 on or off and may be electrically connected to the controller 210 to control supply of the power. In detail, the power switch turns the controller 210 on or off, controls flow of a current from the controller 210 to the vibration motor 220 and the heating part 230, and thus serves to generate or block vibration and heat of the massager 10. FIGS. 1 and 2 illustrate that the power switch is provided in a button shape, but the power switch may be implemented in various shapes as long as the power may be controlled with those shapes.

The controller 210 is a component for controlling the microcurrent, a vibration function, and a heating function of the massager 10 by supplying a current to the protrusion part 110, the vibration motor 220, and the heating part 230.

In detail, the controller 210 is electrically connected to the protrusion part 110 and supplies a current to the first protrusion 111 and the second protrusion 112, and thus the first protrusion 111 and the second protrusion 112 apply the microcurrent to the contact area while in contact with the body.

Meanwhile, the controller 210 may include a sensor that measures the intensity of the current applied to the protrusion part 110 and may adjust the microcurrent applied to the contact area by controlling the intensity of a voltage. In detail, the controller 210 may compare a current value measured from the sensor based on a pre-stored reference current value to calculate an area of the contact area in contact with the protrusion part 110 and may control the intensity of the voltage according to the area of the contact area. In more detail, as an area in which the first protrusion 111 and the second protrusion 112 are in contact with the body increases, the intensity of the current applied to the first protrusion 111 and the second protrusion 112 decreases due to a resistance value of the contact area. In this case, the controller 210 may compare the current value applied to the first protrusion 111 and the second protrusion 112, which is measured by the sensor, with a reference current value and adjust the intensity of the voltage according to the area of the contact area, and thus the first protrusion 111 and the second protrusion 112 may apply the microcurrent having a certain intensity.

Further, the controller 210 may apply power to the vibration motor 220 and the heating part 230 to generate vibration and heat, may massage the contact area through the first protrusion 111 and the second protrusion 112, and at the same time, may transfer heat to help with recovery of the skin and muscles.

The vibration motor 220 is a component for generating vibration to stimulate and massage the contact area with a constant pressure, and at the same time, remove a residual liquid material with which the upper surface of the massager 10 is coated. In detail, the vibration motor 220 may be electrically connected to the controller 210 to receive power from the controller 210 to generate vibration. As the vibration generated by the vibration motor 220 is transmitted, the first protrusion 111 and the second protrusion 112 may stimulate the contact area with a constant pressure. The residual liquid material with which the upper surface or an inner surface of the massager 10 is coated may be removed, and thus hygiene problems can be prevented.

The heating part 230 is a component for generating heat to promote the absorption of the liquid material and preventing the liquid material remaining on the upper surface or the inside of the massager 10 from solidifying. In detail, the heating part 230 may include a heating wire, may be electrically connected to the controller 210, and may generate heat in the heating wire by the power applied from the controller 210. Alternatively, the heating part 230 may include a plurality of light emitting diode (LED) elements, and the plurality of LED elements may be installed on at least one of a first printed circuit board (PCB) substrate and a second PCB substrate so as to radiate light to generate heat.

Meanwhile, the massager 10 may further include an LED module (not illustrated) that promotes the absorption of the liquid material by radiating light in various wavelength bands. Preferably, the LED module may include a plurality of LED light sources, the LED light sources may be spaced a predetermined interval from the protrusion part 110 and provided on the upper surface of the massager 10, and the plurality of LED light sources may emit light having of a specific wavelength, radiate the light to the contact area, and promote the absorption of the liquid material.

The coupling part 300 is a component for coupling or separating the massager 10 to or from the container 20 and transferring the liquid material from the container 20.

The coupling part 300 may include the guide part 310 that is connected to the container 20 and deliver the liquid material from the container 20, and the guide part 310 may be connected to the outlet 101 through the body part 200 so that the outlet 101 may discharge the liquid material.

Exemplary embodiments of the present invention have been disclosed above for purposes of illustration, but those skilled in the art to which the present invention pertains can deduce various modifications, changes, and additions without departing from the spirit and scope of the present invention, and it should be understood that such modifications, changes, and additions belong to the scope of the appended claims.

Since those skilled in the art to which the present invention pertains can deduce various substitutions, modifications, and changes without departing from the technical spirit of the present invention, the present invention is not limited by the above-described embodiments or the accompanying drawings.

### INDUSTRIAL APPLICABILITY

According to the present invention, an active ingredient contained in a liquid material is absorbed deeply into the skin through electrical stimulation using a microcurrent, and thus an effect thereof can be maximized.

According to the present invention, a contact area is stimulated with a constant pressure through vibration in a process of applying and absorbing a liquid material, and thus a massage effect can be increased.

According to the present invention, a residual liquid material present on an upper surface or the inside of a massager through vibration or heating is removed, and thus hygiene problems can be prevented.

1. A massage device including a massager that discharges a liquid material and a container in which the liquid material is accommodated, the massage device comprising:
a protrusion part including a plurality of first protrusions each having an upper end that is partially exposed through an upper surface of the massager and generates a microcurrent and a lower end that is provided inside the massager to have a first height from the upper surface and a plurality of second protrusions each having an upper end that is partially exposed through the upper surface of the massager and generates a microcurrent and a lower end that is provided inside the massager to have a second height from the upper surface;
a first conductor having a plate shape, which is provided in contact with the lower ends of the plurality of first protrusions and transmits a current supplied from a controller to the plurality of first protrusions; and
a second conductor having a plate shape, which is provided in contact with the lower ends of the plurality of second protrusions and transmits a current supplied from the controller to the plurality of second protrusions,
wherein the first height and the second height are different heights.

2. The massage device of claim 1, comprising a separation layer that is provided between the first conductor and the second conductor and insulates the first conductor from the second conductor while maintaining a constant gap between the first conductor and the second conductor.

3. The massage device of claim 1, wherein the controller includes a sensor that measures an intensity of a current applied to the protrusion part,
compares a current value measured by the sensor based on a pre-stored reference current value to calculate an area of a contact area in contact with the protrusion part including the plurality of protrusions, and
controls an intensity of a voltage according to the area of the contact area.

4. The massage device of claim 1, comprising a vibration motor that generates vibration to stimulate a contact area with a constant pressure and removes a residual liquid material with which an upper surface of the massager is coated after use of the massager.

5. The massage device of claim 1, further comprising a heating part that generates heat to promote absorption of the liquid material and prevents the liquid material remaining on an upper surface or an inside of the massager from solidifying after use of the massager.

6. The massage device of claim 1, wherein the first height is greater than the second height.

7. The massage device of claim 1, wherein a diameter of the first protrusion is greater than a diameter of the second protrusion.

8. The massage device of claim 1, wherein an outlet through which the liquid material is discharged is formed on an upper surface of the massager, and
the massager includes a guide part having a tubular shape, which passes through an inside of the massager, is connected to the container, and delivers the liquid material from the container to the outlet.
